(19) United States
(12) Patent Application Publication
(10) Pub. No.: US 2015/0157589 A1
(43) Pub. Date: Jun. 11, 2015

(54) COMPOSITIONS COMPRISING PYRUVATE ALKYL ESTERS AND USES THEREOF

(71) Applicant: Ranya L. Alexander, San Diego, CA (US)

(72) Inventor: Ranya L. Alexander, San Diego, CA (US)

(21) Appl. No.: 14/624,854

(22) Filed: Feb. 18, 2015

Related U.S. Application Data
(62) Division of application No. 12/296,527, filed on Nov. 2, 2009.
(60) Provisional application No. 60/744,739, filed on Apr. 12, 2006.

(51) Int. Cl. A61K 31/22; A23L 1/30; A23L 2/52; A61K 45/06

(52) U.S. Cl. CPC A61K 31/22; A61K 45/06; A23L 1/30; A23L 2/52; A23V 2002/00

(57) ABSTRACT

The present invention provides a composition comprising pyruvic acid alkyl ester for ingestion by a human as a food, oral supplement or pharmaceutical.

### BACKGROUND OF THE INVENTION

The present invention relates generally to the field of compositions for oral supplementation for energy and cellular enhancement, and methods of modulating a variety of human conditions including, without limitation, psychological and physical energy level, mood, physical performance, weight control, hydration, metabolism, thirst control, blood glucose, and cellular production of free radicals. Specifically, the present invention relates to oral compositions comprising alkyl esters of pyruvic acid, for example without limitation, ethylpyruvate, and to methods of use thereof.

Ethylpyruvate occurs naturally in a variety of products, including beer. The compound is generally recognized as safe by the United State Food and Drug Administration as a synthetic flavoring substance. “Synthetic flavoring substance” refers to substances and adjuvants which may be safely used in food in accordance with the following conditions: (a) they are used in the minimum quantity required to produce their intended effect, and otherwise in accordance with all the principles of good manufacturing practice; and (b) they consist of one or more of the compounds listed in 21 C.F.R. §172.515, used alone or in combination with flavoring substances and adjuvants generally recognized as safe in food, prior-sanctioned for such use, or regulated by an appropriate section of 21 C.F.R. §172.

U.S. Pat. No. 4,158,057 reports a medical method of preventing excessive accumulation of fatty deposits in the liver of a mammal due to ingestion of ethanol, which method includes administering orally to the mammal a mixture of pyruvate and dihydroxyacetone, optionally riboflavin.

U.S. Pat. No. 5,294,641 reports a medical method for increasing the cardiac output of a human without concurrently increasing the cardiac oxygen demand of the human which comprises includes feeding the human, orally or intravenously, pyruvate, optionally as an organic ester.

U.S. Pat. No. 5,480,909 reports a medical method for inhibiting free-radical generation and concurrently scavenging internally generated-free radicals in a mammal by administering to the mammal pyruvate, or a pharmaceutically acceptable ester thereof.

U.S. Pat. No. 5,801,198 reports a medical method for retarding loss of morphology in the bowel of a mammal experiencing ischemic bowel by introducing pyruvate, or organic ester thereof, enterally or parenterally into the mammal prior to and during the ischemic bowel or during reperfusion.

U.S. Pat. No. 6,846,842 describes a medical method for treating a mammal suffering from ischemia or reperfusion injury by administering an ester of a 2 ketoalkanoic acid selected from the group consisting of ethyl pyruvate, propyl pyruvate, butyl pyruvate, carboxymethyl pyruvate, acetoxymethyl pyruvate, carbethoxymethyl pyruvate and ethoxymethyl pyruvate in a pharmaceutically-acceptable carrier, wherein the carrier further comprises an organic or inorganic cation.

Additional uses for pyruvate administration have been reported in, e.g.,: U.S. Pat. No. 4,874,790 (medical use of pyruvate for diabetes treatment; U.S. Pat. Nos. 4,812,479, 4,548,937 and 4,351,835 (medical use in retarding weight gain); U.S. Pat. No. 4,415,576 (medical use to increase body protein concentration in a mammal; U.S. Pat. No. 4,315,835 (use for extending athletic endurance; U.S. Pat. No. 5,134,162 (medical use for retarding cholesterol increase; and U.S. application Ser. No. 08/194.857, filed Feb. 14, 1994 (medical use for inhibiting growth in spread of malignancy and retarding DNA breaks).

### SUMMARY OF THE INVENTION

In accordance with the present invention, a variety of benefits as described herein accompany the ingestion of alkyl esters of pyrilvic acid, for example without limitation ethylpyruvate, by a human.

In a first aspect, the invention provides a composition for ingestion which is a food, dietary supplement or pharmaceutical, which composition comprises pyruvic acid alkyl ester, wherein the pyruvic acid alkyl ester is present at greater than 0.01 weight-percent. In a preferred embodiment, the composition for ingestion is a food or dietary supplement rather than a pharmaceutical. In certain embodiments of this aspect, the pyruvic acid alkyl ester is ethylpyruvate.

Without wishing to be bound by any specific scientific theory, it is believed that pyruvate esters, and in particular ethylpyruvate, are sufficiently lipophilic to be taken up by cells at a faster rate than equimolar amounts of pyruvic acid per se or any tautomeric or charged forms thereof. Accordingly, pyruvate esters in the compositions of the present invention serve as carriers for intracellular pyruvate delivery made bioavailable after non-specific ester solvolysis by ubiquitous cytosolic carboxyesterases. Pyruvate oxidation connects glycolysis to the tricarboxylic acid cycle whereby pyruvate molecules formed during glycolysis are transported from the cytoplasm through the mitochondrial membranes into the mitochondrial matrix, wherein the pyruvate dehydrogenase complex catalyzes known biochemical reactions which result in the production of acetyl-CoA. Accordingly, direct provision of pyruvic acid circumvents the need for production of pyruvic acid via glycolysis for cellular energy.

“Ingestion” refers in the context of compositions and methods of the present invention to the taking of a composition into the body via the alimentary canal.

“Dietary supplement” refers to a product as defined under the United States Dietary Supplement Health and Education Act of 1994, which product is intended to supplement the diet and bears or contains one or more of a vitamin, a mineral; an herb or other botanical (excluding tobacco), an amino acid, a dietary substance for use by man to supplement the diet by increasing the total dietary intake, or a concentrate, metabolite, constituent, extract or combination of any of these components. A dietary supplement is intended for ingestion in pill, capsule, tablet, powder or liquid form, and is not represented for use as a conventional food or as the sole item of a meal or diet labeled as a “dietary supplement.” The terms “oral supplement” and “dietary supplement” are used interchangeably herein.

“Pyruvic acid alkyl ester,” “alkyl ester of pyruvic acid” and like terms refer to compounds of Formula I, and all tautomeric and charged forms thereof,

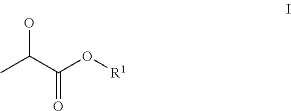

wherein R¹ is alkyl. As well known to one of skill in the art, compounds of Formula I can undergo enolization to form enols of Formula II, which enols can deprotonate to compounds of Formula III. Accordingly, such tautomeric and charged forms are contemplated by the term alkyl ester of pyruvic acid. Furthermore, with respect to pyruvic acid, the terms “pyruvic acid,” “pyruvate” and like chemical terms are used interchangeably, all referring to the various tautomeric and charge states of pyruvic acid.

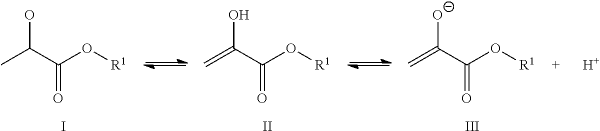

The term “alkyl” refers to straight, branched chain, or cyclic hydrocarbyl groups including from 1 to about 20 carbon atoms. Alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. Preferred alkyl groups include alkyl groups having from 1 to 6 carbon atoms, more preferred alkyl groups have 2 carbon atoms (i.e., ethylpyruvate). It is understood that all compositions and methods provided by the present invention which comprise pyruvic acid alkyl esters include any of the alkyl esters specified above and most preferably ethylpyruvate.

“Weight-percent,” the symbol “wt-%” and equivalent terms refer to the ratio of the mass of a specified component of a composition to the total mass of the composition, expressed as a percentage. Accordingly, 0.1 weight-percent is equivalent to 1.0 gm/kg (i.e., 1000 parts per million), 0.01 weight-percent is equivalent to 0.1 gm/kg (i.e., 100 parts per million), and so forth.

“Pharmaceutical” and like terms refer to compounds, for example drugs, which have been found effective to treat and/or ameliorate one or more specific diseases for which the compound is approved, and which meet safety criteria, well known to those of skill in the art, by being subject to extensive animal and/or controlled human testing. Pharmaceuticals are typically administered in accordance with a medical method under the direction of trained medical personnel.

“Medical method” and like terms refer to a method of treatment conducted under the auspices of a trained medical practitioner. Compounds and compositions employed in medical methods are pharmaceuticals as defined herein.

The pyruvic acid alkyl ester used in the compositions of the invention is preferably ethylpyruvate. The term “ethylpyruvate” contemplates all tautomeric and charged forms of the compound according to any of Formulae I-III having R¹ equal to ethyl. In some embodiments, the nutritional content afforded by ethylpyruvate as an oral supplement accounts for 0.1-50% of the daily nutritional needs of the subject. In some embodiments, the nutritional content afforded is within another range, e.g., 0.1-40%, 0.1-30%, 0.1-20%, 0.1-10%, 0.1-5%, 0.1-1%.

In another aspect, the invention provides methods for the energy enhancement of a human upon ingesting an effective amount of a composition of the invention comprising a pyruvic acid alkyl ester as defined herein. In some embodiments, the pyruvic acid alkyl ester is ethylpyruvate.

“Energy enhancement” refers to a subjective feeling by a human subject that the strength, stamina and/or inclination to undertake a task has been increased by ingestion of a composition of the invention. An energy enhancement may be accompanied by feelings that additional need for energy is not required. For example without limitation, energy enhancement may accompany a feeling by a human subject that additional stimulation e.g., ingesting a caffeine containing food or beverage, is not necessary in the contemplation of a task. Thus, by ingesting an effective amount of a composition of the invention, energy enhancement is achieved, which may be associated with reduced intake of stimulants such as caffeine.

The term “effective amount” and like terms indicate that the amount of material is effective to achieve the desired result, for example without limitation, energy enhancement in a human. Accordingly, an effective amount of a composition according to the present invention may contemplate a variety of ranges of amount of pyruvic acid alkyl ester, depending on the human subject, including for example without limitation, 100 mg to 100 gm, 100 mg to 200 gm, 100 mg to 500 gm, and specific amounts therein, e.g.: about 100 mg, 200 mg, 500 mg, 1 gm, 2, gm, 3 gm, 4 gm, 5 gm, 10 gm, 15 gm, 20 gm, 30 gm, 40 gm, 50 gm, 100 gm, 200 gm, 300 gm, 400 gm or even 500 gm. These amounts can be delivered by multiple administrations over a period of time.

“Pharmaceutically acceptable” and like terms in the context of formulations and composition indicates that the specified composition does not have properties that would cause a reasonably prudent medical practitioner to avoid administration of the composition to a patient, taking into consideration the disease or conditions to be treated and the respective route of administration.

In another aspect, the invention provides methods for the enhancement of physical performance by a human upon ingesting an effective amount of a composition of the invention comprising a pyruvic acid alkyl ester as defined herein. In some embodiments, the pyruvic acid alkyl ester is ethylpyruvate.

“Physical performance” refers to objective indicia of physical activity, as well known in the art, and to a subjective feeling or belief by a human subject that greater physical ability attends the ingestion of a composition according to the present invention.

In another aspect, the invention provides a method for promoting weight loss in a human, which method comprising ingesting an effective amount of a composition of the invention comprising a pyruvic acid alkyl ester as defined herein. In some embodiments, the pyruvic acid alkyl ester is ethylpyruvate. Benefits of the invention may be afforded by oral ingestion of the pyruvic acid alkyl ester to account for a specific percentage of daily nutritional need, as well known in the art, for example without limitation, 1%, 2%, 3%, 4%, 5%, 10%, 15%, 20%, 25%, 30%, 40%, 50% or even higher.

In another aspect, the invention provides a method for mood enhancement in a human, the method comprising oral ingestion of a compound of the invention comprising a pyruvic acid alkyl ester as defined herein. In some embodiments, the pyruvic acid alkyl ester is ethylpyruvate. “Mood” and like terms in the context of human feelings refer to a subjective self-assessment of a human, for example without limitation, feelings of being tired, normal, energetic, excited, and the like, all readily understood. Accordingly, “mood enhancement” and like terms refer to an improvement (e.g., more elevated, typically happier or more satisfied) in mood following ingestion of the composition.

In another aspect, the invention provides a method for enhancement of physical performance in a human, the method comprising oral ingestion of a composition of the invention comprising a pyruvic acid alkyl ester as defined herein. In some embodiments, the pyruvic acid alkyl ester is ethylpyruvate. “Enhancement of physical performance” refers to both an objective increase in, for example and without limitation, strength and stamina of a human, as judged by physiological methods well known in the art, and additional to a subjective increase in feelings of well-being and potential to undertake a physical task.

In another aspect, the invention provides a method for re-hydration or thirst quenching of a human, the method comprising oral ingestion of an aqueous (e.g., beverage) composition of the invention comprising a pyruvic acid alkyl ester as defined herein. In some embodiments, the pyruvic acid alkyl ester is ethylpyruvate.

In another aspect, the invention provides a method for metabolism enhancement in a human, the method comprising oral ingestion of a composition of the invention comprising a pyruvic acid alkyl ester as defined herein. In some embodiments, the pyruvic acid alkyl ester is ethylpyruvate. Without wishing to be bound by theory, it is believed that provision of pyruvic acid resulting from ester hydrolysis of an alkyl ester thereof (e.g., ethylpyruvate) provided by a composition of the invention facilitates the increase in flux through the tricarboxylic acid cycle, thereby providing for enhancement of metabolism.

In another aspect, the invention provides a method for supplementing, via an oral supplement, the actions of intravenous infusion of ethylpyruvate for resuscitation of cardiac, muscle or nerve tissue, the method comprising oral ingestion of a composition according to the present invention.

In another aspect, the invention provides a method for increasing fat metabolism, and thereby decreasing depot fat, in a human, the method comprising oral ingestion of an effective amount of a composition according to the invention comprising a pyruvic acid alkyl ester as defined herein. In some embodiments, the pyruvic acid alkyl ester is ethylpyruvate. Without wishing to be bound by theory, it is believed that provision of pyruvic acid resulting from ester hydrolysis of an alkyl ester thereof (e.g., ethylpyruvate) provided by a composition of the invention facilitates the increase in flux through the tricarboxylic acid cycle, thereby decreasing the need for glycolysis (i.e., glucose utilization) and upregulating the cellular machinery available to metabolize fat, thereby resulting in increased fat utilization and decrease in depot fat.

In another aspect, the invention provides a method for decreasing blood glucose levels in a human, the method comprising oral ingestion of an effective amount of a composition according to the invention comprising a pyruvic acid alkyl ester as defined herein. In some embodiments, the pyruvic acid alkyl ester is ethylpyruvate. Accordingly, reduction in blood glucose may be afforded by oral ingestion of an alkyl ester of pyruvic acid to account for a specific percentage of daily nutritional need, as well known in the art, for example without limitation, 0.1%, 1%, 2%, 3%, 4%, 5%, 10%, 15%, 20%, 25%, 30%, 40%, 50% or even higher.

In another aspect, the invention provides a method for inhibition of the cellular production of free radicals in a human, the method comprising oral ingestion of an effective amount of a composition according to the invention comprising a pyruvic acid alkyl ester as defined herein. In some embodiments, the pyruvic acid alkyl ester is ethylpyruvate. Accordingly, reduction in cellular production of free radicals may be afforded by oral ingestion of an alkyl ester of pyruvic acid to account for a specific percentage of daily nutritional need, as well known in the art, for example without limitation, 1%, 2%, 3%, 4%, 5%, 10%, 15%, 20%, 25%, 30%, 40%, 50% or even higher. Without wishing to be bound by theory, it is believed that pyruvic acid per se has anti-oxidant properties, for example, pyruvate has been reported as a free radical scavenger (see, e.g., De Boer, L. W. V. et al, Am. J. Physiol 265 (Heart Circ. Physiol 341): H1571-H11576, 1993).

In another aspect, the invention provides a method for improvement of cognitive, language, behavior or social skills of an autistic human, the method comprising oral ingestion of an effective amount of a composition according to the invention comprising a pyruvic acid alkyl ester as defined herein. In some embodiments, the pyruvic acid alkyl ester is ethylpyruvate.

In another aspect, the invention provides a method for enhancing the recovery of a human from a disease, the disease selected from the group consisting of mononucleosis, chronic fatigue syndrome, and viral infection, the method comprising repeated oral ingestion of an effective amount of a composition according to the invention comprising a pyruvic acid alkyl ester as defined herein. In some embodiments, the pyruvic acid alkyl ester is ethylpyruvate.

In another aspect, the invention provides a food additive comprising greater than 0.01 weight-percent alkyl ester of pyruvic acid. In some embodiments, the food additive comprises greater than 0.01 weight-percent ethylpyruvate.

### DETAILED DESCRIPTION OF THE PREFERRED EMBODIMENTS

In certain embodiments of compositions according to the present invention, the amount of pyruvic acid alkyl ester (e.g., ethylpyruvate) is present in the range greater than 0.01 to 30 weight-percent. In other embodiments, the range is greater than 0.01 to about 30 weight-percent. “About” in the context of numeric values and ranges indicates the nominal value+/−10%. In certain embodiments, the amount of pyruvic acid alkyl ester (e.g., ethylpyruvate) is present in the following approximate ranges (i.e., +/−10%): 0.02-0.05, 0.05-0.1, 0.1-1, 1-2, 2-5, 5-10, 10-20, or 20-30 weight-percent. In certain embodiments, the amount of pyruvic acid alkyl ester (e.g., ethylpyruvate) is present in specific amounts, e.g., about 0.02, 0.05, 0.1, 1, 2, 3, 4, 5, 6, 7, 8, 9, 10, 15, 20, 25 or even 30 weight-percent.

In certain embodiments, the present invention provides compositions for oral ingestion comprising pyruvic acid alkyl ester, and in particular ethylpyruvate, wherein the composition lacks sodium or calcium. In certain embodiments, the present invention provides compositions for oral ingestion comprising pyruvic acid alkyl ester, and in particular ethylpyruvate, wherein the composition further comprises sodium, calcium, and/or magnesium.

In certain embodiments, the present invention provides compositions for oral ingestion comprising pyruvic acid alkyl ester, and in particular ethylpyruvate, wherein the compositions are in the form of food. As used herein, a “food” is a nutritious solid, semi-solid, liquid, food ingredient or food additive. “Semi-liquid” refers in the context of food to an otherwise solid component dispersed in a liquid milieu, e.g. without limitation, cereal in milk. A food bar or candy bar comprising a pyruvic acid alkyl ester as defined herein (e.g. ethylpyruvate) is an exemplary solid food composition of the invention.

In certain embodiments, the compositions provided by the invention are foods or dietary supplements in the form of a beverage. In certain embodiments, the compositions are foods. In certain embodiments, the compositions are dietary supplements.

A liquid or beverage composition for oral ingestion can be taken cold (e.g., with ice), at room temperature, or after heating to a convenient temperature (e.g., 80-120 degrees F.) depending on the needs of the human.

In certain embodiments, the present invention provides a food additive or food ingredient comprising a pyruvate alkyl ester. “Food additive” or “food ingredient” refers to substances which are not typically ingested per se, but which are used in the preparation of food and/or beverages to achieve the benefits provided by the compositions of the invention. Examples of food additive or ingredient include, without limitation, a concentrated form of a composition according to the present invention for mixing with a beverage or food component during preparation thereof.

In certain embodiments, compositions provided by the present invention further comprise one or more components selected from the group consisting of flavorings, colorings, non-carbohydrate sweeteners, vitamins, electrolytes, Coenzyme Q₁₀, aloe, minerals, fulvic acid, mushrooms, dissolved effervescent gas, amino acids, carbohydrates, stimulants, proteins, herbs, essential oils and extracts.

In certain embodiments of methods contemplated by the invention, a single ingestion of a composition provided herein is sufficient to elicit a beneficial response. For example without limitation, increase in physical performance, enhancement of mood, re-hydration, thirst quenching, metabolic enhancement, enhancement of fat metabolism, decrease in blood glucose, and inhibition of cellular production of radical chemical species, can be observed upon ingestion of a single portion of a composition of the invention. In certain embodiments, the single portion provides the nutritional content equivalent to 0.1-50% of the daily nutritional needs of the subject. In some embodiments, the nutritional content afforded is within another range, e.g., 0.1-40%, 0.1-30%, 0.1-20%, 0.1-10%, 0.1-5%. Advantageously, rapid onset of beneficial effects, for example without limitation, within 1, 2, 3, 4, 5, 10, 15, or as little as 20 min, is afforded in methods contemplating oral ingestion of alkyl esters of pyruvic acid (e.g., ethylpyruvate) according to the invention. As used herein, a single portion of a composition of the invention is generally a volume of fluid or solid than can be ingested in one sitting, which sitting may last from less than a minute to several minutes and generally up to less than an hour. Exemplary single portions can include 1-4 oz solid bar or an 8-32 oz drink.

In certain embodiments of methods contemplated by the invention, repeated ingestion of a composition provided herein facilitates a beneficial response. For example without limitation, promotion of weight loss, supplementation of the action of intravenous infusion of pharmaceuticals (e.g., ethylpyruvate or other alkyl ester of pyruvic acid delivered via a non-oral mode), increased fat metabolism, and enhanced recovery from a disease selected from the group consisting of mononucleosis, chronic fatigue syndrome, and viral infection, can be observed upon repeated ingestion of portions of a composition of the invention over an extended period of time (e.g., days to weeks). In certain embodiments, the portions contemplated for repeated ingestion provide nutritional content equivalent to 0.1-50% of the daily nutritional needs of the subject. In some embodiments, the nutritional content afforded is within another range, e.g., 0.1-40%, 0.1-30%, 0.1-20%, 0.1-10%, 0.1-5%.

### EXAMPLE 1

### Stability

It has been observed that ethylpyruvate in solution demonstrates higher stability at room temperature (e.g., about 50 to 90 degrees F.) than does the methyl ester of pyruvic acid, or corresponding tautomeric and/or charged forms thereof, as judged by appearance (e.g., color) and taste. Without wishing to be bound by theory, it is believed that methyl esters of pyruvic acid are more susceptible to hydrolysis in aqueous solution than are corresponding ethyl esters, which hydrolysis results in the production of pyruvic acid. Pyruvic acid, and the anion thereof, can then react by mechanisms well known in the art, to form a plethora of higher molecular compounds. Accordingly, the higher stability of ethylpyruvate results in longer shelf life for compositions of the present invention, as compared with corresponding compositions employing the methyl ester of pyruvic acid.

### EXAMPLE 2

### Taste Assessment

A panel of 10 individuals ingested a liquid composition (about 30 ml) at room temperature comprising ethylpyruvate (about 1 weight-percent) to provide a taste assessment. The panel was additionally provided a corresponding liquid composition of methylpyruvate (i.e., methyl ester of pyruvic acid) for comparative tasting. Each of the individuals judged the methylpyruvate solution to be “sour.” In contrast, each of the individuals judged the ethylpyruvate solution to have the taste associated with Granny Smith apples. Without wishing to be bound by theory, it is believed that the sour taste associated with methypyruvate results from hydrolysis of methylpyruvate within the sample to pyruvic acid, with the associated source taste of an acid. In contrast, the taste associated with ethylpyruvate appears to reflect a lack of hydrolysis to pyruvic acid in the sample.

### EXAMPLE 3

### Gastric Reflux

In the taste assessment experiment (Example 2), gastric reflux (i.e., “heartburn”) was a considerable problem (i.e., 5 of 10 individuals reporting such) with methylpyruvate, but none reporting similar reaction with ethylpyruvate. Without wishing to be bound by any theory, it is believed that the free pyruvic acid in the methylpyruvate sample, as a result of ester hydrolysis prior to ingestion, resulted in pyruvic acid eliciting a gastric reflux reaction.

### EXAMPLE 4

### Mood Enhancement

In the taste assessment experiment (Example 2), mood was assessed before and after ingestion of the liquid composition comprising ethylpyruvate. Each of the individuals reported an increase in feelings of well-being and energy after ingesting the composition. This mood enhancement commenced at between 5 and 15 minutes and lasted over 6 hours. In one case, the individual stated that the usual afternoon cup of coffee would not be necessary that day due to the ingestion of the composition.

### EXAMPLE 5

### Exemplary Compositions

Exemplary compositions of the present invention could include a carbonated beverage comprising a pyruvate alkyl ester (e.g., ethylpyruvate), optionally added vitamins and minerals, to provide a refreshing alternative to soft drinks. Methods for formulation of such carbonated beverages are well known in the art. Exemplary vitamin and mineral components include vitamins B3, B6, B12; exemplary minerals include zinc, sodium, magnesium, and calcium.

Further exemplary compositions could include candy bars or energy bars comprising a pyruvate alkyl ester (e.g., ethylpyruvate). Methods of manufacture of candy bars are well known in the art (see e.g., U.S. Pat. No. 4,491,597). In candy bars contemplated by the present invention, the pyruvate alkyl ester (e.g., ethylpyruvate) can be suspended or dissolved in a candy matrix forming the interior of the candy bar, or suspended or dissolved in an optional coating, as known in the art.

Further exemplary compositions could include suspensions or solutions of a pyruvate alkyl ester (e.g., ethylpyruvate) in combination with shaved, granulated, crushed, or pulverized ice to afford, for example without limitation, “snow cones” or “slush” drinks comprising ethylpyruvate. As is well known, snow cones are made by depositing flaked ice into a cone shaped cup and applying a flavoring syrup thereon (see U.S. Pat. No. 4,174,742). As further well known in the art, slush drinks comprise ice and flavorings and are typically dispensed at the point of sale with specialized machinery, well known in the art. Accordingly, the present invention contemplates snow cones or slush drinks to which pyruvate alkyl ester (e.g., ethylpyruvate) has been added.

Further exemplary compositions could include chewing gums or soups to which pyruvate alkyl ester (e.g., ethylpyruvate) has been added.

### EXAMPLE 6

### Post-Surgical Oral Ingestion of Ethylpyruvate

Patient response to ingestion of a composition according to the invention was observed in a patient following surgery and radiation therapy for gastrointestinal carcinoma. The patient had developed gastrointestinal inflammation and could not tolerate food by mouth for several days post-operative. Furthermore, bowel movements were frequent, watery and bloody, and were accompanied by lower abdominal pain. The patient self administered oral ethylpyruvate (about 3×500 mL per day daily for 1 week of an ethylpyruvate solution in commercial bottled spring water at 0.06 weight-percent ethylpyruvate). Within 3 hours of the initial dosing, persistent pain, hyperactive bowel and diarrhea resolved. The patient additionally reported an improved mood following oral ingestion of ethylpyruvate.

### EXAMPLE 7

### Athlete Oral Ingestion of Ethylpyruvate

A group of 12 athletes (19-22 years of age) self-administered oral ethylpyruvate in solution (4×500 mL ingested per day of a solution of about 0.06 weight-percent ethylpyruvate in water) before and during daily workouts (approximately 2 hours duration) for four days. The athletes having ingested oral ethylpyruvate reported less fatigue at the end of each workout, and similar favorable reports continued each day for the four days of practice. Each subject additionally reported better performance in high-activity training regimens (e.g., “wind sprints” as known in the art) relative to performance reported in practice sessions prior to oral ingestion of ethylpyruvate.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually. Also incorporated by reference in its entirety and for all purposes is U.S. Provisional Application No. 60/744,739, filed Apr. 12, 2006.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, and defined by the scope of the claims.

It will be readily apparent to one skilled in the art that varying substitutions and modifications may be made to the invention disclosed herein without departing from the scope and spirit of the invention. For example, various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. Thus, for an embodiment of the invention using one of the terms, the invention also includes another embodiment wherein one of these terms is replaced with another of these terms. In each embodiment, the terms have their established meaning. Thus, for example, one embodiment may encompass a method “comprising” a series of steps, another embodiment would encompass a method “consisting essentially of” the same steps, and a third embodiment would encompass a method “consisting of” the same steps. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

1-10. (canceled)

11. A method for enhancing energy or reducing fatigue of a human individual, comprising orally administering to the human individual a food or dietary supplement comprising from 0.01 to 30 weight-percent ethylpyruvate.

12. A method for enhancing the physical performance of a human athlete, comprising orally administering to the human athlete a food or dietary supplement comprising from 0.01 to 30 weight-percent ethylpyruvate.

13. (canceled)

14. A method for improving the mood of a human individual, comprising orally administering to the human individual a food or dietary supplement comprising from 0.01 to 30 weight-percent ethylpyruvate.

15-25. (canceled)

26. The method of claim 11, wherein the food or dietary supplement comprises from 0.02 to 5 weight-percent ethylpyruvate.

27. The method of claim 11, wherein the food or dietary supplement comprises from 0.05 to 0.1 weight-percent ethylpyruvate.

28. The method of claim 11, wherein the food or dietary supplement comprises from 0.1 to 1 weight-percent ethylpyruvate.

29. The method of claim 11, wherein the food or dietary supplement comprises from 1 to 2 weight-percent ethylpyruvate.

30. The method of claim 11, wherein the food or dietary supplement is a beverage.

31. The method of claim 11, wherein the food or dietary supplement is a solid bar.

32. The method of claim 11, wherein the food or dietary supplement further comprises one or more other components selected from the group consisting of flavorings, colorings, non-carbohydrate sweeteners, vitamins, electrolytes, Coenzyme Q10, aloe, minerals, fulvic acid, mushrooms, dissolved effervescent gas, amino acids, carbohydrates, stimulants, proteins, herbs, essential oils and extracts.

33. The method of claim 12, wherein the food or dietary supplement comprises from 0.02 to 5 weight-percent ethylpyruvate.

34. The method of claim 12, wherein the food or dietary supplement is a beverage.

35. The method of claim 12, wherein the food or dietary supplement is a solid bar.

36. The method of claim 12, wherein the food or dietary supplement further comprises one or more other components selected from the group consisting of flavorings, colorings, non-carbohydrate sweeteners, vitamins, electrolytes, Coenzyme Q10, aloe, minerals, fulvic acid, mushrooms, dissolved effervescent gas, amino acids, carbohydrates, stimulants, proteins, herbs, essential oils and extracts.

37. The method of claim 14, wherein the food or dietary supplement comprises from 0.02 to 5 weight-percent ethylpyruvate.

38. The method of claim 14, wherein the food or dietary supplement is a beverage.

39. The method of claim 14, wherein the food or dietary supplement is a solid bar.

40. The method of claim 14, wherein the food or dietary supplement further comprises one or more other components selected from the group consisting of flavorings, colorings, non-carbohydrate sweeteners, vitamins, electrolytes, Coenzyme Q10, aloe, minerals, fulvic acid, mushrooms, dissolved effervescent gas, amino acids, carbohydrates, stimulants, proteins, herbs, essential oils and extracts.
